(19) United States
(12) Patent Application Publication
(10) Pub. No.: US 2025/0000476 A1
(43) Pub. Date: Jan. 2, 2025

(54) ANATOMIC VISUALIZATION AND MEASUREMENT FOR ORTHOPEDIC SURGERIES

(71) Applicant: Hologic, Inc., Marlborough, MA (US)

(72) Inventor: Marc HANSROUL, Wynnewood, PA (US)

(73) Assignee: Hologic, Inc., Marlborough, MA (US)

(21) Appl. No.: 18/292,491

(22) PCT Filed: Aug. 4, 2022
(86) PCT No.: PCT/US2022/039459

§ 371 Date: Jan. 26, 2024

Related U.S. Application Data
(60) Provisional application No. 63/229,375, filed on Aug. 4, 2021.

(51) Int. Cl. A61B 6/58; A61B 6/00; A61B 6/46; A61B 6/50

(52) U.S. Cl. CPC A61B 6/589; A61B 6/4405; A61B 6/4441; A61B 6/463; A61B 6/505; A61B 6/5205

(57) ABSTRACT

A system and method for determination of one or more dimensions (e.g., width or length) of an anatomic feature (e.g., bone) of a subject includes a radiographic imaging system configured to image the subject, an optical imaging system computer to determine the position of the subject relative to one or more components of the radiographic imaging system, and one or more processors configured to determine the one or more dimension of the anatomic feature based on an image, generated by the radiographic imaging system, of the anatomic feature and the determined position of the subject relative to one or more components of the radiographic imaging system.

This application is being filed on Aug. 4, 2022, as a PCT International Patent Application and claims the benefit of priority to U.S. Provisional Patent Application Ser. No. 63/229,375, filed Aug. 4, 2021, the entire disclosure of which is incorporated by reference in its entirety.

### INTRODUCTION

Accurate measurements and depiction of anatomic features, such as bones, are important to many medical procedures, such as orthopedic surgery. For example, in fracture reduction procedures, orthopedic surgeons need to have precise bone dimensions in order to select fracture reduction hardware of optimal sizes. Traditional estimation of bone dimensions by visual observation or invasive procedures often results in imprecise sizing and adds complexity and uncertainty to surgical workflow. While fiducial markers can sometimes be used in medical imaging as scale indicators, they are difficult to position on certain parts of the human body, such as limbs, and are difficult to use when the subject does not remain stationary. Efforts are ongoing in developing systems and methods for accurate and convenient measurements of anatomic features.

### SUMMARY

In one aspect, the example systems devices, and methods described in the present disclosure are related to imaging and real-time measurement of anatomic features. In some embodiments, a system for measuring a dimension, such as the length or width, of an anatomic feature includes: a first imaging system including a radiation source configured to emit radiation to a subject containing the anatomic feature, a radiation detector configured to receive radiation from the subject in response to the radiation emitted from the radiation source to the subject and to generate signals indicative of a special distribution of an attribute of the received radiation, and one or more processors configured to receive the signals from the radiation detector and generate image data corresponding to an image of the anatomic feature based on the received signals. The system further includes a second imaging system configured to acquire information relating to a relative position between the subject and the radiation source or radiation detector. The one or more processors are adapted to derive one or more dimensions of the anatomic feature based on the relative position between the subject and the radiation source or radiation detector.

In some embodiments, the first imaging system is an x-ray imaging system, including: an x-ray source, an x-ray detector (e.g., a flat-panel x-ray detector), and a processing unit. The x-ray source and x-ray detector are disposed to accommodate a subject (e.g., a human hand or a portion of a human arm) between them; the x-ray source is configured to emit x-ray toward the subject; the x-ray detector is configured to receive x-ray emitted from x-ray source and passed through the subject and generate signals indicative of the attenuation of the x-ray by different portions of the subject; and the processor unit is programmed to receive the signal from the x-ray detector and generate an image of the subject, including the anatomic feature in the subject, within the field-of-view. In some embodiments, the second imaging system includes a first optical camera, such as an infrared depth camera disposed in a known position relative to the x-ray imaging system (e.g., at the x-ray source of an x-ray imaging system). The camera is configured to measure the distance between the subject and the camera, thereby determining the relative position between the subject and the x-ray source or x-ray detector of the x-ray imaging system. In some embodiments, the processing unit is further configured to derive one or more dimensions (e.g., width or length) of the anatomic feature based on the size of the image of the anatomic feature and the relative position between the subject and the x-ray source or x-ray detector of the x-ray imaging system.

In some embodiments the second imaging system further includes a second optical camera configured to acquire optical images of the subject. Alternatively, the first optical camera can be configured to acquire optical images of the subject in addition to measuring distance. The optical images in some embodiments are combined with the images generated by the first imaging system (e.g., x-ray imaging system) to create composite images. Such composite images can be used to create an augmented reality (AR) environment, which can be utilized to facilitate surgical procedure planning, surgery guidance, surgical team communication, and educational communications. In some embodiments, certain foreign objects of high radiographic contrast, such as metallic fracture reduction hardware, can be identified from the optical images (e.g., by machine vision), and the regions of high radiographic contrast anomalies created by such foreign objects are excluded from automatic contrast/brightness adjustment in generating radiographic images of the anatomic features.

In some embodiments, a method for measuring a dimension of an anatomic feature of the subject includes: acquiring a radiographic image of the anatomic feature using a radiographic imaging system; determining, substantially simultaneously with the acquisition of the radiographic image, and using an optical imaging system, the position of the subject relative to one or more components of the radiographic imaging system; and determining the dimension of the anatomic feature based on the radiographic image and the position of the subject relative to one or more components of the radiographic imaging system. In some embodiments, the position of the subject relative to one or more components of the radiographic imaging system is defined by the distance between the subject and the radiation source or detector of the radiographic imaging system. In some embodiments, the determination of the dimension of the anatomic feature includes determining the size of the image of the anatomic feature in the radiographic image, and adjusting the size of the image of the anatomic feature by a magnification factor, which in some embodiments is a function of the distance between the subject and one or more components of the radiographic imaging system, and of the distance between two or more components of the radiographic imaging system.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

The same number represents the same element or same type of element in all drawings.

FIG. 1 depicts a scanning system with a 3D optical camera in accordance with one embodiment of the technology.

FIG. 2 depicts a scanning system with a 3D optical camera in accordance with another embodiment of the technology.

FIG. 3A depicts an example embodiment of an X-ray source module including a 3D optical camera in accordance with one or more features of the present disclosure; the source module including the camera may be used in connection with the C-arm assembly shown in FIG. 1 or 2 in accordance with an embodiment of the technology.

FIG. 3B depicts another example embodiment of an X-ray source module including a 3D optical camera in accordance with one or more features of the present disclosure; the source module including the camera may be used in connection with the C-arm assembly shown in FIG. 1 or 2 in accordance with an embodiment of the technology.

FIG. 4 depicts a schematic illustration of an imaging system with a 3D optical camera in accordance with one or more features of the present disclosure; the magnification factor by which the dimension of an anatomic feature in an object is enlarged in the image acquired by the imaging system is determined by the distance between the subject and the radiation source or detector of the imaging system in accordance with an embodiment of the technology.

FIG. 5 depicts a method of determining the thickness of the scanning target in accordance with an embodiment of the technology.

FIG. 6 illustrates a surgical setting in which an object is placed in an x-ray imaging apparatus equipped with a device measuring the distance between the x-ray source and the objection, according to some embodiments.

FIGS. 7A and 7B illustrate magnification in x-ray imaging depending on the distance of the object to the x-ray source.

FIGS. 8A and 8B show an optical image and depth map, respectively, of an object. Depth maps are utilized to determine actual dimensions of the objects from x-ray images, according to some embodiments.

FIGS. 9A and 9B show, respectively, an x-ray image of an object and a depth map of the object. The images are co-registered for ascertaining dimensions of features (e.g., bones) of the object from x-ray images, according to some embodiments.

FIG. 10 illustrates a process by which actual dimensions of features of objects are determined from x-ray images of the objects, according to some embodiments.

FIGS. 11A, 11B, and 11C illustrate a process by which the dimension of a feature in an object is determined from an x-ray image of the object, according to some embodiments.

FIG. 12 illustrates a process by which the dimensions of features in an object is determined from an x-ray image of the object, according to some embodiments.

FIGS. 13A through 13H are a series of augmented-reality (AR) images created from an optical image of an object, in this example a patient's hand, and an x-ray image of the object according to some embodiments. The optical image and x-ray image are scaled to each other using real-time measured distance between the x-ray source and the object and are superimposed on one another to create the AR images. The contrasts of the optical image and the x-ray image are weighted differently for each of the AR images, with the weight of the contrast of the x-ray image increasing progressively relative to the weight of the contrast of the optical image from the AR image in FIG. 13A to the AR image in FIG. 13H.

FIGS. 14A, 14B, and 14C show surgical planning for a small bone external fixator based on an AR image of a patient's hand, according to some embodiments, and optical and x-ray images of the patient's hand with the fixator applied, respectively.

FIG. 15 shows a frame of video display showing corresponding x-ray and optical images of an object, according to some embodiments.

### DETAILED DESCRIPTION

The present technology provides real-time measurements of anthropometric measurements of anatomic features imaged with radiographic imaging systems. In some embodiments a first (e.g., radiographic) imaging system and a second (e.g., optical) imaging system are integrated, with the first imaging system configured to acquire x-ray images of anatomic features (e.g., bones) of the subject (e.g., hand), and the second imaging system determining the distance between the subject being imaged by a radiographic imaging system and certain components (e.g., radiation source and a radiation detector), and/or acquiring optical images of the subject. The distance in some embodiments is used to establish a relationship between the measurements of anatomic features in the x-ray images and the actual dimensions of the anatomic features. The actual dimensions can thus be derived from the radiographic images regardless of the position of the subject relative to the imaging systems (e.g., the distance between a subject and the source or detector of the radiographic imaging system).

In some embodiments, the first imaging system is a conventional x-ray fluoroscopy system, such as the mini c-arm imaging system disclosed in the U.S. Pat. No. 9,161,727, issued Oct. 20, 2015 (the “U.S. '727 patent”), to Jenkins et al. and commonly assigned with the present application. An example fluoroscopic system disclosed in the U.S. '727 patent includes an x-ray source assembly and an image detector assembly, which can include a flat panel detector (FPD). The x-ray source assembly and image detector assembly are mounted on a mini C-arm assembly. The mini C-arm assembly may be mounted to a mobile base via an articulating arm assembly for supporting the mini C-arm in a selected position. The computer processing and a user interface (e.g., keyboard, monitor etc.) may also be mounted on the mobile base. The C-arm is carried by the support arm assembly such that a track of the C-arm is slidable within the C-arm locking mechanism. The x-ray source assembly and x-ray detector assembly are respectively mounted at opposite extremities of the C-arm in facing relation to each other so that an x-ray beam from x-ray source assembly impinges on the input end of the detector assembly. The x-ray source assembly and detector end are spaced apart by the C-arm sufficiently to define a gap between them, in which the limb or extremity of a human patient can be inserted in the path of the x-ray beam. The support arm assembly provides three-way pivotal mounting that enables the C-arm to swivel or rotate through 360° in each of three mutually perpendicular (x, y, z) planes and to be held stably at any desired position, while the articulating arm assembly is mounted to the portable cabinet and jointed to enable the C-arm to be angularly displaced both horizontally and vertically. The multidirectional angular movability of the C-arm assembly facilitates the positioning of the x-ray source and detector assemblies in relation to a patient body portion to be irradiated.

Another x-ray imaging system is disclosed in the international patent application PCT/US2021/036619, filed under the Patent Cooperation Treaty on Jun. 9, 2021 (the “PCT '619 application”) and published as International Publication No. WO 2022/139874 A1 (the “WO '874 Publication”), and commonly assigned with the present application. Certain details of an example mini C-arm fluoroscope disclosed in the '619 application are described below.

The disclosures of the U.S. '727 patent and PCT '619 application are incorporated herein by reference in their entirety.

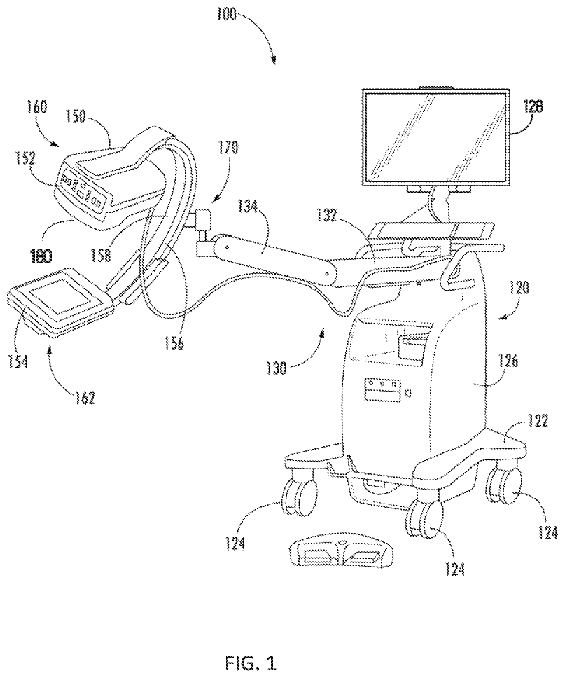

Referring to FIG. 1, a system for determining one or more dimensions of an anatomic feature imaged by an x-ray imaging system includes an x-ray imaging system 100 and an 3D optical camera, such as a depth-sensing camera 180. The x-ray imaging system (a mini C-arm) 100 is described in detail in the PCT '619 application. As illustrated, the mini C-arm 100 includes a base 120, a C-arm assembly 150, and an arm assembly 130 for coupling the C-arm assembly 150 to the base 120. As illustrated, the base 120 may include a platform 122 and a plurality of wheels 124 extending from a bottom surface of the platform 122 so that the base 120, and hence the mini C-arm 100, can be movably located by the operator as desired. The wheels 124 are selectably lockable by the operator so that when in a locked state, the wheels 124 allow the operator to manipulate the arm assembly 130 without shifting the location or orientation of the base 120. The base 120 may also include a cabinet 126. As will be appreciated by one of ordinary skill in the art, the cabinet 126 may be sized and configured for storing, for example, controls (not shown) for operating the mini C-arm 100, electrical components (not shown) needed for operation of the mini C-arm 100, counterweights (not shown) needed to balance extension of the C-arm assembly 150, a brake system, a cord wrap, etc. The cabinet 126 may also include, for example, a keyboard, one or more displays (such as a monitor 128), a printer, etc.

The arm assembly 130 may include a first arm 132 and a second arm 134, although it is envisioned that the arm assembly 130 may include a lesser or greater number of arms such as, for example, one, three, four, etc. The arm assembly 130 enables variable placement of the C-arm assembly 150 relative to the base 120. As illustrated in the exemplary embodiment, the arm assembly 130, and more specifically the first arm 132, may be coupled to the base 120 via a vertically adjustable connection. Other mechanisms for coupling the arm assembly 130 to the base 120 are also envisioned including, for example, a pivotable connection mechanism. The second arm 134 may be coupled to the first arm 132 via a joint assembly to enable the second arm 134 to move relative to the first arm 132. In addition, the second arm 134 may be coupled to the C-arm assembly 150 via an orbital mount 170, as will be described in greater detail below. Thus arranged, the arm assembly 130 enables the C-arm assembly 150 to be movably positioned relative to the base 120.

As previously mentioned, the mini C-arm 100 also includes a C-arm assembly 150. The C-arm assembly 150 includes a source 152, a detector 154, and an intermediate body portion 156 for coupling to the source 152 and the detector 154. As will be readily known by one of ordinary skill in the art, the imaging components (e.g., X-ray source 152 and detector 154) receive photons and convert the photons/X-rays to a manipulable electrical signal that is transmitted to an image processing unit (not shown). The image processing unit may be any suitable hardware and/or software system, now known or hereafter developed to receive the electrical signal and to convert the electrical signal into an image. Next, the image may be continuously acquired and displayed on a display that may be a monitor or TV screen (e.g., monitor 128). The image can also be stored, printed, etc. The image may be a single image or a plurality of images.

The intermediate body portion 156 of the C-arm assembly 150 includes a curved or arcuate configuration. For example, the intermediate body portion 156 may have a substantially “C” or “U” shape, although other shapes are envisioned. The intermediate body portion 156 includes a body portion 158 and first and second end portions 160, 162 for coupling to the source and detector 152, 154, respectively. In certain embodiments, the body portion 158 and the first and second ends 160, 162 of the intermediate body portion 156 may be integrally formed. Alternatively, these portions of the intermediate body portion 156 can be separately formed and coupled together. The X-ray source 152 and the detector 154 are typically mounted on opposing ends of the C-arm assembly 150 and are in fixed relationship relative to each other. The X-ray source 152 and the detector 154 are spaced apart by the C-arm assembly 150 sufficiently to define a gap between them in which the patient's anatomy can be inserted in the path of the Xray beam. As illustrated, the C-arm assembly 150 may include an orbital mount 170 for coupling the C-arm assembly 150 to the arm assembly 130. The body portion 158 rotates or orbits relative to the orbital mount 170 to provide versatility in positioning the imaging components relative to the portion of the patient's anatomy to be irradiated.

The 3D optical depth-sensing camera 180 is integrated into, or attached to, the x-ray source 152.

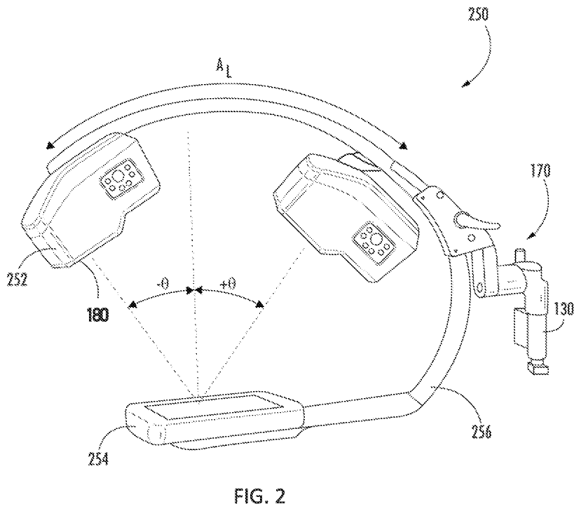

Referring to FIG. 2, a system for determining one or more dimensions of an anatomic feature imaged by an x-ray imaging system includes an x-ray imaging system and an optical depth-sensing camera 180. The x-ray imaging system (a mini C-arm) is described in detail in the PCT '619 application. As illustrated, the mini C-arm may include a C-arm assembly 250 including a source 252, a detector 254, and an intermediate body portion 256 wherein the source 252 is movable along an arc length AL of the intermediate body portion 256 of the C-arm assembly 250. In one embodiment, the C-arm assembly 250 may enable±θ degrees of movement of the source 252 relative to the detector 254. In one example embodiment, θ may be equal to 45 degrees.

In addition, and/or alternatively, the source may move in a plane transverse to the arc length AL. In either event, the source 252 may be repositioned to, for example, enable the operator to acquire multiple images of the patient's anatomy without movement of the detector 254. More specifically, by arranging the source 252 to move along the arc length AL of the intermediate body portion 256 of the C-arm assembly 250 (and/or transverse thereto), the surgeon can acquire multiple views of the patient's anatomy including, for example, an anterior-posterior view or a posteroanterior view (PA) and an oblique or lateral view without moving the patient's anatomy from the detector 254.

The source 252 may be movably coupled to the intermediate body portion 256 of the C-arm assembly 250 via any known mechanism for movably coupling the source 252 to the C-arm assembly 250. For example, in one example embodiment, the source 252 may be coupled to the intermediate body portion 256 of the C-arm assembly 250 via a track that extends along an arc length AL thereof. The source 252 may be coupled to the track so that the source 252 can be moved, repositioned, etc., along the track, which extends along the arc length AL of the intermediate body portion 256 of the C-arm assembly 250.

In one embodiment, the source 252 may be manually positionable along the arc length AL of the intermediate body portion 256 of the C-arm assembly 250. For example, in one embodiment, the source 252 may slide along the arc length AL of the intermediate body portion 256 of the C-arm assembly 250. In one embodiment, the source 252 can be continuously movable along an arc length AL of the intermediate body portion 256 of the C-arm assembly 250. Alternatively, the source 252 may be positionable at predefined angles, positions, etc.

Alternatively, and/or in addition, in one embodiment, the source 252 may be moved relative to the intermediate body portion 256 of the C-arm assembly 250 via, for example, motorized control. For example, the mini C-arm may include a motor to move the source 252 along an arc length AL of the intermediate body portion 256 of the C-arm assembly 250. For example, in one embodiment, the source 252 may be coupled to the intermediate body portion 256 of the C-arm assembly 250 via a connector unit, which may house a motor operatively coupled to an output gear, which may be operatively coupled to a drive system such as, for example, a drive belt and pulley system, a lead screw drive system, or the like. Activation of the motor rotates the output gear, which rotates the belt about the pulleys, which moves the source.

The 3D optical depth-sensing camera 180 is integrated into, or attached to, the movable x-ray source 252.

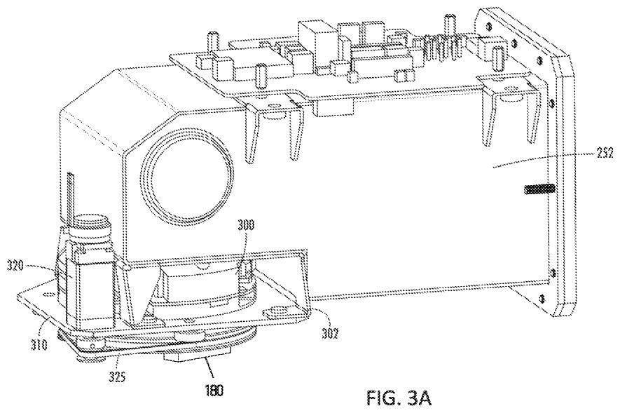

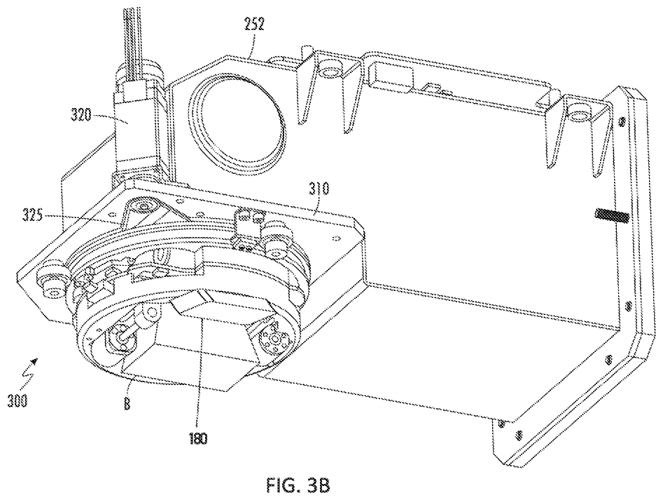

Referring to FIGS. 3A and 3B, a system for determining one or more dimensions of an anatomic feature imaged by an x-ray imaging system includes an x-ray imaging system, including the aperture assemblies 300 of the x-ray source 252, and an optical depth-sensing camera 180. The aperture assemblies 300 of the x-ray imaging system 252 are described in detail in the PCT '619 application. As illustrated, the aperture assembly 300 is coupled to the X-ray source or source module such as, for example, the X-ray source 252 shown in FIG. 2. As illustrated, the aperture assembly 300 receives the X-ray beam B from the source 252 and enables the X-ray beam B to pass therethrough. As will be discussed in greater detail below, the aperture assembly 300 may alter, modify, etc. the shape of the X-ray beam B transmitted from the source 252 and passing through the aperture assembly 300, which in turn alters the beam area (field of view) projected onto the detector surface.

The aperture assembly 300 may be coupled to the source 252 via any suitable mechanism and/or method now known or hereafter developed. For example, in one embodiment, as illustrated in FIG. 3A, the source 252 may include a bracket 302 for coupling to a mounting plate 310 of the aperture assembly 300 via one or more fasteners. As illustrated, in the embodiment of FIG. 3A, the coupling mechanism (e.g., bracket and fastener) may be a bottom mount design (e.g., the bracket 302 is coupled to a mounting plate 310 positioned at or adjacent to a bottom portion of the aperture assembly 300). Alternatively, for example, referring to FIG. 3B, the aperture assembly 300 may include a mounting plate 310 for coupling to the source 252 via one or more fasteners. As illustrated, in the embodiment of FIG. 3B, the mounting plate 310 may be positioned at or adjacent to an upper end of the aperture assembly 300 thus the aperture assembly 300 may be referred to as a top mount design.

The aperture assembly 300 may be rotatably coupled to the source 252. The aperture assembly 300 may be rotatably coupled to the source 252 via any suitable mechanism and/or method now known or hereafter developed. For example, in one embodiment, the aperture assembly 300 may include a motor 320 operatively coupled to the aperture assembly 300 so that activation of the motor 320 rotates the aperture assembly 300. For example, as illustrated, the motor 320 may be coupled to the aperture assembly 300 via a drive belt and pulley system 325. However, alternate mechanisms for coupling the motor 320 to the aperture assembly 300 are envisioned. For example, the motor may be coupled to the aperture assembly via a gear driven system. The optical depth-sensing camera 180 is integrated into, or attached to, the aperture assembly 300.

The optical depth-sensing camera 180 can be of a variety of types, including structured light and coded light, stereo depth, and the time-of-flight (TOF) and LIDAR. Structured light cameras project patterns of light onto objects and analyze distortions in the reflected light. The reflected light patterns can reveal the proximity, motion, or contours of the object. A time-of-flight sensor measures the time it takes for light to travel from the sensor an object and back, and the distance from the sensor and the object is determined from the measured time. Active stereo vision combines two or more cameras, such as near-infrared cameras, to triangulate the location and movements of an object. To perform these measurements, in some examples, depth-sensing cameras include their own light sources. In some examples, such light sources are laser sources integrated into a device near the camera. Infrared light sources are often used for this purpose.

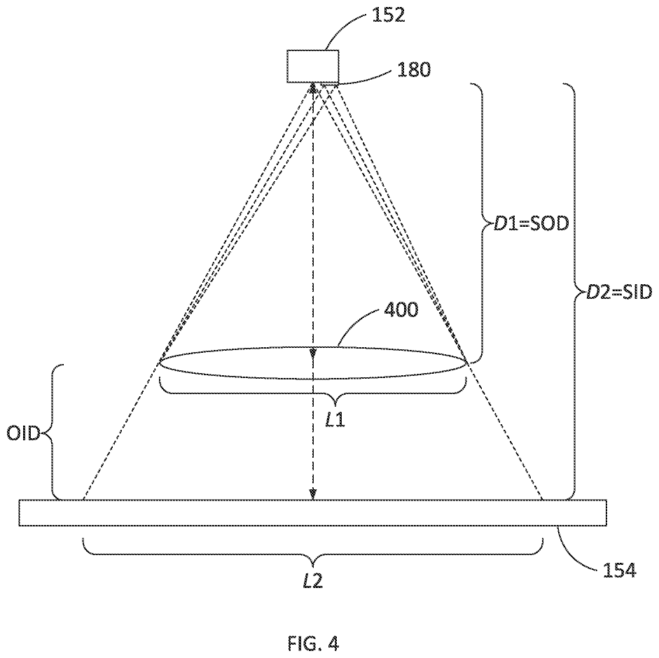

With the optical camera 180 mounted at the x-ray source, as shown in FIG. 4, the camera 180 is configured to measure the distance D1 (source-object distance or SOD)) between the subject 400 imaged by the x-ray imaging system and the x-ray source 152. A magnification factor, M, which is the ratio between the size L2 of an image of the subject and the actual dimension L1 of the actual subject, is given by L2/L1=SOD/SID, where SID is the source-image distance. The actual dimension (e.g., the width of length) of an anatomic feature (e.g., bone) is thus given by (dimension of anatomic feature in the x-ray image)/M.

The capability of capturing real-world lengths, widths, and heights of anatomic features with more clarity and in-depth detail than thus can be achieved with the embodiments disclosed above. In some embodiments, user interfaces (UIs) can be configured to provide ease selecting parts of the anatomy to be measured. For example, touch screen or voice commands UIs can be used to select regions-of-interest (ROIs) or portions of the anatomy in which dimension measurements are to be carried out.

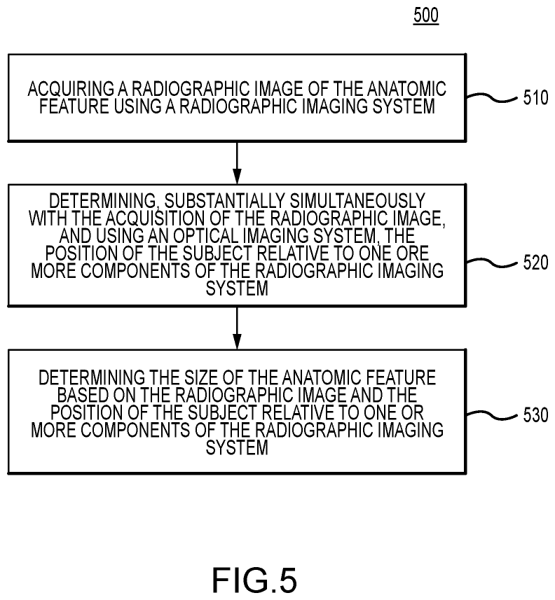

Referring to FIG. 5, in some embodiments, a method 500 for measuring one or more dimensions of an anatomic feature of the subject includes: acquiring a radiographic image of the anatomic feature using a radiographic imaging system 510; determining, substantially simultaneously or sequentially, with the acquisition of the radiographic image, and using an optical imaging system, the position of the subject relative to one or more components of the radiographic imaging system 520; and determining the dimension of the anatomic feature based on the radiographic image and the position of the subject relative to one or more components of the radiographic imaging system 530.

In some embodiments, the combined imaging system described herein can be used for augmented reality (AR) applications. In one example, optical camera capabilities are augmented for object recognition. The optical image can be scaled and overlaid with its corresponding radiographic image to create an augmented reality environment. Radiographic data can be used to create a display with more integration and interaction. One application of the AR technology is surgical procedure planning and guidance using both depth maps (distance measurements) and optical images of anatomical objects provided by the 3D sensing method provides.

In some embodiments, artificial intelligence (AI) technology can be incorporated into the imaging/measurement systems to enhance the performance of those systems. For example, certain standard measurements that a system provides to the user may depend on the actual bone which is being measured. For example, scaphoid bone is not measured the same way as a metacarpal bone. AI (e.g., based on unsupervised learning from prior images using artificial neural networks (ANNs)) can be used to recognize (in the radiographic image) which bone is being measured, and appropriate standard dimensions can be automatically selected and displayed on the image. Alternatively, the user can select or input the particular bone of interest, and one or more look-up tables, which can be stored in the processor, can be accessed to derive measurements for the selected bone. More details and examples illustrating certain embodiments and their applications are provided below.

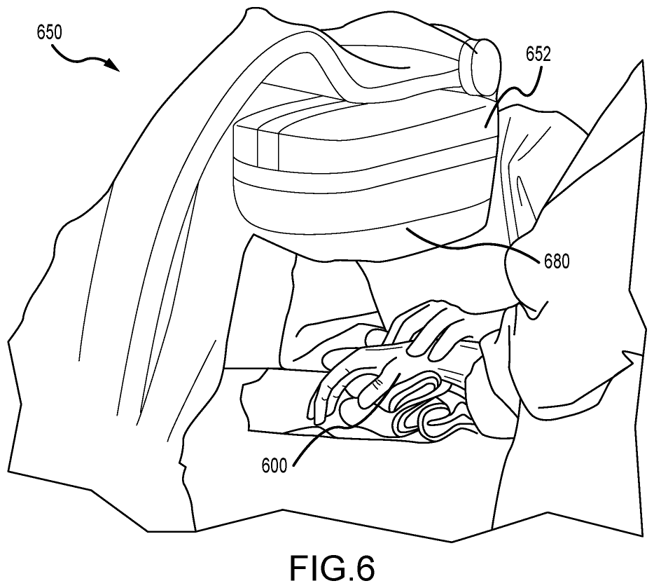

In some embodiments, such as that shown in FIG. 6, in a surgical setting, the subject 600, in this example a patient's hand, is supported inside an x-ray imaging apparatus 650, such as a C-arm assembly. The imaging apparatus 650 includes an x-ray source 652 and an x-ray detector (not shown). In some embodiments, the detector is at a fixed distance from the x-ray source. For example, in a mini c-arm the source to detector distance is equal to or less than 45 cm. The imaging apparatus 650 in this example further includes a depth-sensing camera 680. In some embodiments, the depth-sensing camera 680 is disposed proximate the x-ray source 652. For example, the depth-sensing camera 680 can be integrated into the collimator of the x-ray source 652.

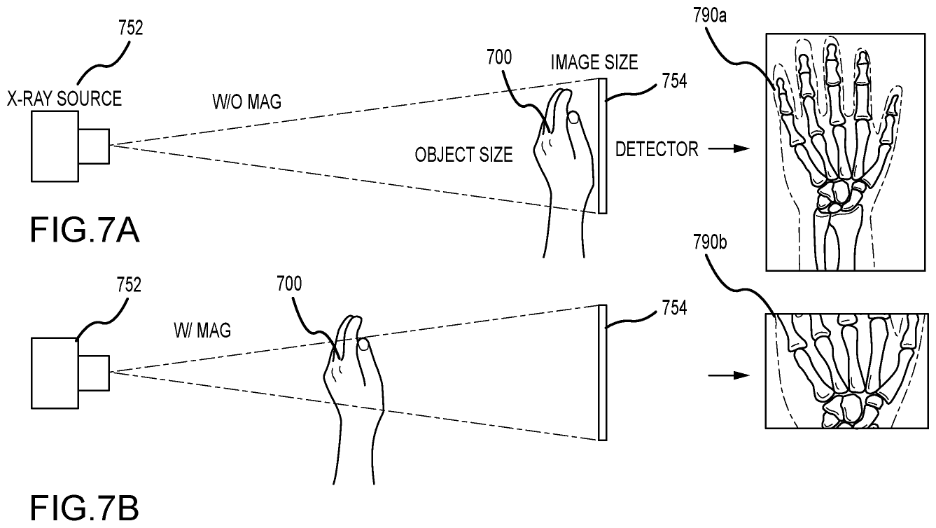

In a setting such the one shown in FIG. 6, the size of the x-ray image of the object (e.g., a patient's hand) depends at least in part on the distance between the x-ray source and the object (SOD)) relative to the distance between the x-ray source and the imaging plane, i.e., the detector (SID)). As illustrated in FIG. 7A, an object 700 is disposed at substantially the same distance from the x-ray source 752 as the detector 754 (i.e., SOD)=SID)), no magnification occurs, and the dimension of the object in the x-ray image 790a is substantially the same as the true dimension of the object 700 (i.e., height, length, and width). In contrast, as illustrated in FIG. 7B, if an object 700 is positioned closer to the x-ray source such that the distance from the object 700 to the x-ray source 752 is smaller than the distance from the source to the detector 754 (i.e., SOD<SID)), the dimension of the object in the x-ray image 790b is the dimension of the object 700 magnified by a factor of SID/SOD. To accurately determine, in real time, the actual dimension of an object, or a part of an object (e.g., a metacarpal bone), with a known SID, the SOD is determined by any suitable device and method, including the aforementioned types of depth-sensing cameras mounted on, or integrated into, the x-ray imaging apparatus.

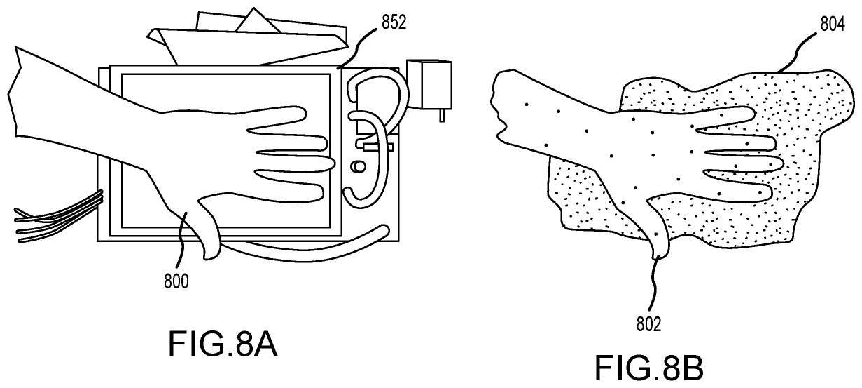

In some embodiments, as illustrated by the example shown in FIGS. 8A and 8B, the distances from the x-ray source to portions of the object, in this case a human hand above the detector (pictured in the optical image in FIG. 8A), are determined in real time with a depth-sensing camera. In some embodiments, the measured distances (depths) are visually presented on the display (e.g., display 128 in FIG. 1) as a depth map, as shown in the example in FIG. 8B, in which distance is represented by a visual characteristic, such as color or contrast.

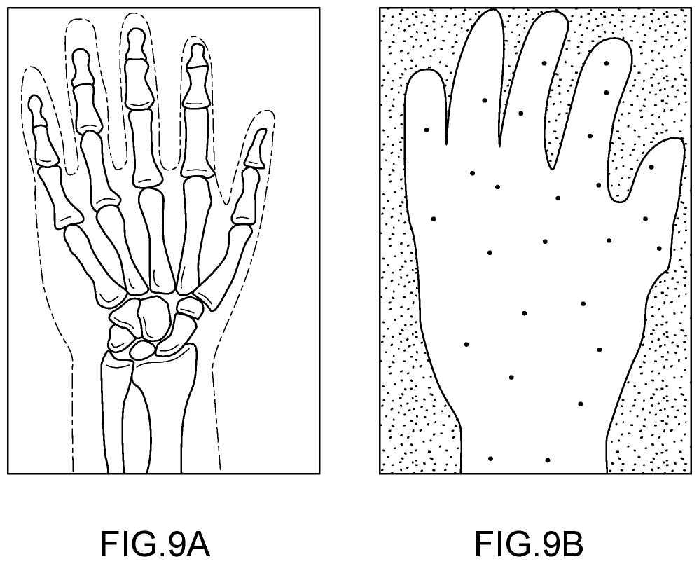

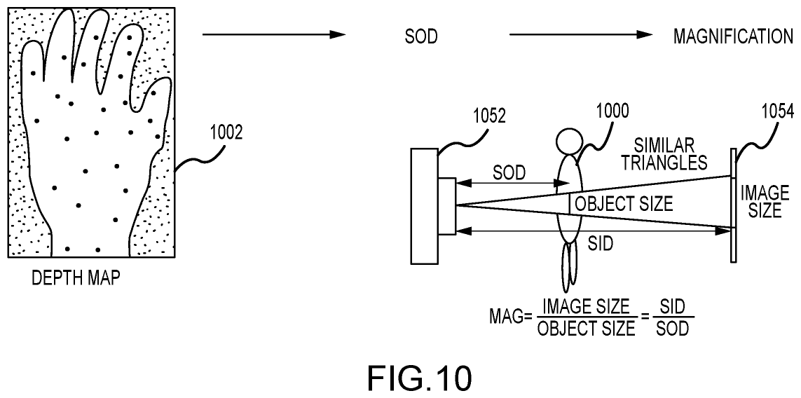

In some embodiments, the x-ray image of an object and the depth map, which can be taken in real time with a depth-sensing camera mounted on, or integrated into, the x-ray imaging apparatus, of the object are co-registered with each other, as shown in FIGS. 9A (x-ray image) and 9B (depth map) so that the distance from the x-ray source to each portion of the object is correlated with the x-ray image of that portion of the object. The x-ray image can be a still image (i.e., a snapshot) of the object or continuously updated (i.e., live video). The dimensions of various features of the object and distances between various features of the object can then be readily determined from the x-ray image. As illustrated in FIG. 10, a depth map 1002 represents the distances (i.e., SOD)) from the x-ray source 1052 to the different portions of the object 1000 being imaged. The magnification factor, M, which is the ratio between the dimension of a given feature in the x-ray image (image dimension) and the actual dimension of the feature (object dimension), is SID/SOD. SID, the distance between the x-ray source 1052 and the x-ray detector 1054, is typically known. For example, in certain types of x-ray imaging apparatuses, such as certain systems with C-arm assemblies, SID) is specified or readily measurable, and is fixed. The magnification factor, M, can then be readily determined.

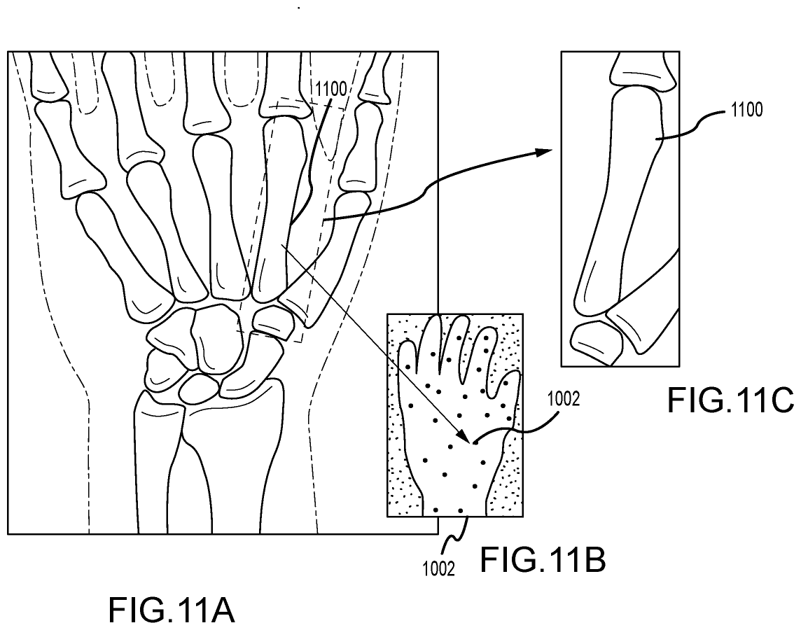

In some embodiments, such as the example shown in FIGS. 11A, 11B, and 11C, the actual dimension of an object is determined in real time from the image dimension of the object and the magnification factor obtained using the SOD, from the depth map, for the object. For example, to determine the width of the second metacarpal bone 1100, the image dimension of the bone is determined as the number of pixels (50 in this example) the width spans in the image times the pixel width (150 μm in this example). From the data for the depth map 1102, the SOD (40 cm in this example) for the bone's location 1104 is determined. Given the SID) (45 cm in this example), the magnification factor, M, is determined by SID/SOD (M=1.125 in this example). The actual bone width is thus readily determined as (image dimension)/M (50×150 μm/1.125=6.6 mm in this example).

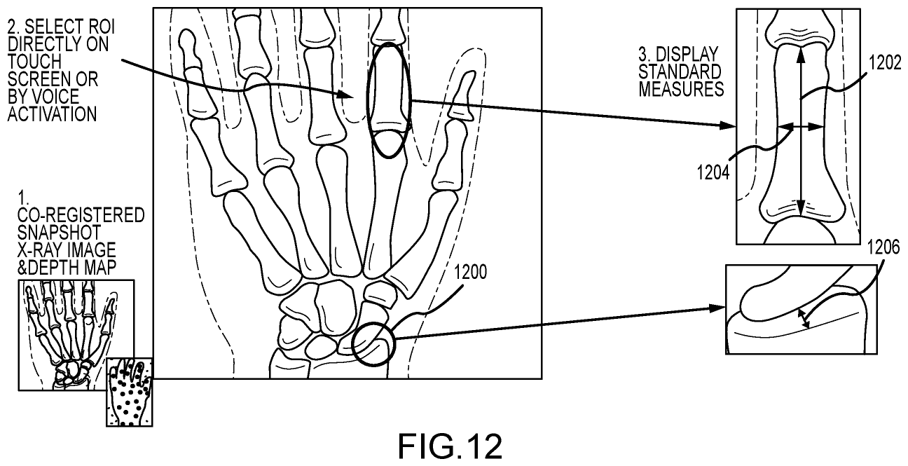

In addition to determining actual dimensions of objects, such as bones, the apparatus and method described about can also be used to measure other distances of interest. For example, as shown in the example in FIG. 12, actual bone dimensions can be measured as described above in reference to FIGS. 11A, 11B, and 11C. In addition, other actual distances of interest, such as the spacing 1200 between bones, can be determined using the same method, i.e., measuring the spacing in the x-ray image and dividing the measured spacing by M. Both the dimensions and distances (e.g., in number of pixels or units of distance) measured in the x-ray image and the actual dimensions and distances can be displayed by, for example, displaying numerical values of the dimensions and distances and visually associating the values with the displayed dimension markers 1202, 1204, 1206, or providing a scale bars on the display.

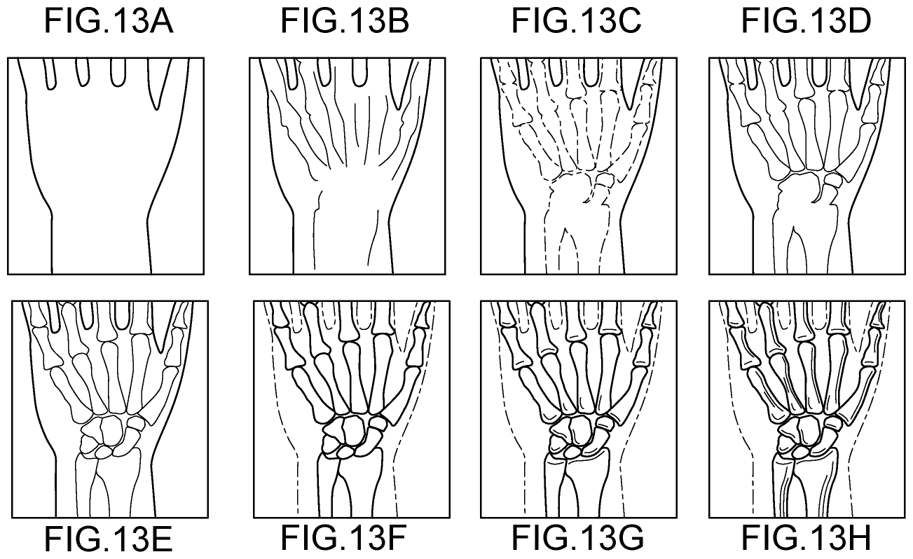

In some embodiments, an x-ray imaging apparatus capable of determining actual object dimensions (such as an x-ray system with a depth-sensing camera) includes additional components, such as an optical camera, to generate AR images for medical procedural planning and guidance. The proposed 3D sensing method provides both depth maps (distance measurements) and optical images of anatomical objects. For example, optical images taken from the same viewpoint as the x-ray source and the corresponding x-ray image can be scaled to match each other, and overlaid on each other to create an augmented-reality environment. With the actual dimensions and distances obtained using the x-ray images and depth-sensing camera, precise planning, such as locations of incision and implants, sizes of implants, and sizes of frames or brackets for fixation, can be made in an AR environment. As an example, shown in FIGS. 13A through 13H, an optical image and the corresponding x-ray image, taken from substantially the same viewpoint, of an anatomic object, such as a hand, are co-registered and overlaid with each other to form an AR image, in which both the external structure (from the optical image) and internal structure (from the x-ray image) are visualized. In the examples shown in FIGS. 13A through 13H, the contrasts of the optical image and the x-ray image are weighted differently for each of the AR images, with the weight of the contrast of the x-ray image increasing progressively relative to the weight of the contrast of the optical image from the AR image in FIG. 13A to the AR image in FIG. 13H. The object (hand in this example) thus appear progressively more transparent in the AR images, from only the external structure of the object being visible in the AR image in FIG. 13A to the internal, skeletal structure being most visible in the AR image in FIG. 13H. The AR images can be presented on a display (e.g., display 128 in FIG. 1) individually, or two or more of the AR images can be presented simultaneously. With the AR images of the object of different apparent transparency, the physician can view the object in the most desirable combination of the x-ray image and the optical image during a surgical procedure.

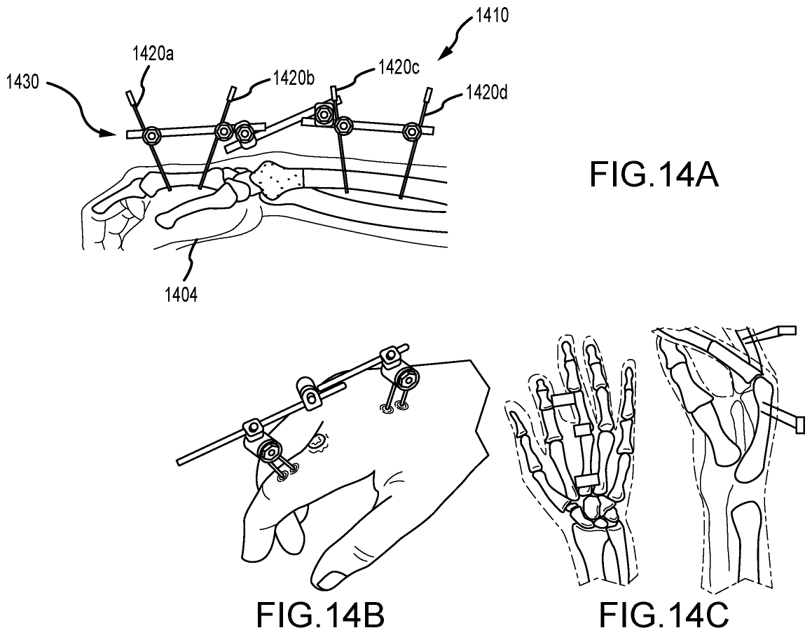

Another example of AR image-aided surgical procedure planning is shown in FIGS. 14A, 14B, and 14C. In this example, as schematically shown in FIG. 14A, an AR image 1404, which combines an optical image and x-ray image as described above in reference to FIGS. 13A and 13B, is used to plan the surgical installation of an external fixator 1410. With the precise knowledge of the bone dimensions and surrounding tissues, the locations of the pins 1420a, 1420b, 1420c, 1420d, the sizes of the pins, and the sizes of the external fixation rods 1430 can be precisely chosen. Actual procedure (FIG. 14B) can be carried out with precision. FIG. 14C shows the result of the application of the external fixator.

In some embodiments, optical images are used in conjunction with automated identification systems to generate enhanced medical images (e.g., x-ray images), in which features of lesser interest are de-emphasized. For example, in certain embodiments, certain foreign objects of high radiographic contrast, such as the metallic external fixator 1410 in FIG. 14A, can be identified from the optical images (e.g., FIG. 14B) by machine vision.

The regions of high radiographic contrast anomalies created by such foreign objects are deemphasized or excluded from automatic contrast/brightness adjustment in generating radiographic images (e.g., FIG. 14C) of the anatomic features. The resulting radiographic images are thus enhanced in that better views of the anatomic features of interest are obtained.

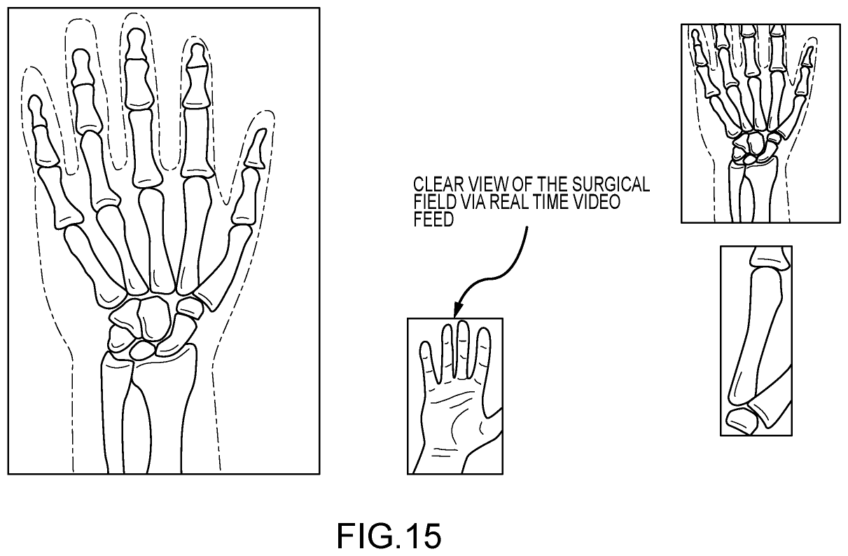

In some embodiments, the one or more optical cameras included in an x-ray imaging apparatus, as described above, is also used to provide a video feed, via a video system (not shown) to a display at one or more locations remote of the surgical site to ease patient positioning or to facilitate communication between the surgeons and other operating staff. Such a capability is particularly advantageous when the operating field is crowded with instruments obstructing direct line of sight of the anatomy. As shown in FIG. 15, one or more video displays can provide a clear view of the surgical field in real time video, as well as x-ray images of the anatomical object placed in the operating field.

With at least certain embodiments, including those examples described above, provide accurate, non-invasive, and often real-time, assessment of dimensions of various anatomic features can be achieved. While non-invasive measurements can be done with certain conventional imaging systems, such measurements often involve the use of fiducials and are difficult to carry out, especially with certain body parts, such as limbs, on which it is difficult to position fiducials, and fiducials tend to crowd the field of view for surgeries; frequent movements of objects, such as limbs, in certain settings, such as surgery, also make the use fiducials impractical. Certain embodiments, including those examples described above, offer advantages over traditional systems and methods. Such advantages include reducing errors in measurements of dimensions of anatomic features, reducing the number of repeated measurements needed, reducing the steps it takes to measure the dimensions. Such reductions result in reduce intervention (e.g., surgery and other medical procedures) time, which in turn results in improved outcome in patient care.

Illustrative examples of the systems and methods described herein are provided below. An embodiment of the system or method described herein may include any one or more, and any combination of, the clauses described below:

Clause 1. A system for determining one or more dimensions of an anatomic feature in a subject, the system comprising:
a first imaging system including a radiation source configured to emit radiation toward a subject containing the anatomic feature, a radiation detector configured to receive radiation in response to the radiation emitted from the radiation source toward the subject and to generate signals indicative of an attribute of the received radiation, and one or more processors configured to receive the signals from the radiation detector and generate image data corresponding to an image of the anatomic feature based on the received signals; and
a second imaging system configured to acquire information relating to a relative position between the subject and the radiation source or radiation detector,
the one or more processors being adapted to derive one or more dimensions of the anatomic feature based on the relative position between the subject and the radiation source or radiation detector.

Clause 2. The system of Clause 1, wherein:
the first imaging system is an x-ray imaging system, the radiation source comprises an x-ray source, and the radiation detector comprises an x-ray detector, the x-ray source and x-ray detector being disposed to accommodate a subject therebetween;
the x-ray source is configured to emit x-ray energy toward the subject;
the x-ray detector is configured to receive x-ray energy emitted from x-ray source and passed through the subject and generate signals indicative of the attenuation of the x-ray energy by different portions of the subject; and the one or processors are programmed to receive the signal from the x-ray detector and generate an image of the subject, including the anatomic feature in the subject, within a field-of-view.

Clause 3. The system of Clause 2, wherein the second imaging system includes an optical camera disposed in a predetermined position relative to the x-ray source, the optical camera being configured to measure the distance between the subject and the optical camera, thereby determining the relative position between the subject and the x-ray source or x-ray detector of the x-ray imaging system.

Clause 4. The system of Clause 3, wherein the one or more processors are further configured to derive one or more dimensions of the anatomic feature based on the size of the image of the anatomic feature and the relative position between the subject and the x-ray source or x-ray detector of the x-ray imaging system acquired from the second imaging system.

Clause 5. The system of any of Clauses 1-4, wherein the second imaging system includes a second optical camera configured to acquire optical images of the subject.

Clause 6. The system of Clause 2 or 3, further comprising an x-ray collimator for the x-ray source, wherein the optical camera is disposed proximate the x-ray collimator.

Clause 7. The system of any of Clauses 1-4, wherein the x-ray imaging system comprises a C-arm x-ray imaging system and further comprises:
a base; and
a C-arm having two extremities and a mid-section between the two extremities, the C-arm being supported by the base at the mid-section, wherein the x-ray source and x-ray detector are supported at the respective extremities of the C-arm.

Clause 8. The system of Clause 6, wherein the one or more processors are configured combine the optical images with the images generated by the first imaging system to create composite images.

Clause 9. A method for measuring a one or more dimensions of an anatomic feature of a subject, the method including:
acquiring a radiographic image of the anatomic feature using a radiographic imaging system;
determining, substantially simultaneously with the acquisition of the radiographic image, and using an optical imaging system, the position of the subject relative to one or more components of the radiographic imaging system; and
determining the one or more dimensions of the anatomic feature based on the radiographic image and the position of the subject relative to one or more components of the radiographic imaging system.

Clause 10. The method of Clause 9, wherein the position of the subject relative to one or more components of the radiographic imaging system is measured based on the distance between the subject and the radiation source or detector of the radiographic imaging system.

Clause 11. The method of Clause 9 or 10, wherein the determination of the one or more dimensions of the anatomic feature includes determining the size of the image of the anatomic feature in the radiographic image, and adjusting the size of the image of the anatomic feature by a magnification factor, which is a function of the distance between the subject and one or more components of the radiographic imaging system and of the distance between two or more components of the radiographic imaging system.

Clause 12. The system of Clause 1, wherein the first imaging system comprises an x-ray imaging apparatus, and the second imaging system comprises a depth-sensing camera disposed and adapted to provide a measure of a source-to-object distance (SOD) between the radiation source and the subject.

Clause 13. The system of Clause 12, wherein the radiation detector is spaced apart from the radiation source by a source-to-image distance (SID), and the one or more processors are adapted to derive one or more dimensions of the anatomic feature based on SOD, SID, and a size of the image of the anatomic feature.

Clause 14. The system of Clause 5, wherein the one or more processors are adapted to generate image data corresponding to a composite image of the optical image and the image based on the received signals from the x-ray imaging apparatus.

Clause 15. The system of Clause 5, further comprising a display adapted to display in real time the optical image acquired by the second optical camera and the image based on the received signals from the x-ray imaging apparatus.

Clause 16. The system of Clause 11, wherein the depth-sensing camera is adapted to generate a map indicative of distances from the radiation source to respective portions of the subject being imaged.

Clause 17. The system of Clause 16, wherein the one or more processors are further adapted to co-register the map with the image based on the received signals from the x-ray imaging apparatus.

Clause 18. A system for determining a one or more dimensions of an anatomic feature in a subject, the system comprising:
a base;
a C-arm having two extremities and a mid-section between the two extremities, the C-arm being supported by the base at the mid-section,
an x-ray source supported at a first one of the extremities of the C-arm;
an x-ray detector supported at a second one of the extremities of the C-arm, the x-ray source and x-ray detector being spaced apart to accommodate a subject therebetween the x-ray source being configured to emit x-ray radiation toward the x-ray detector, the x-ray detector being configured to receive the x-ray radiation from the x-ray source and generate signals indicative of an attribute of the received radiation;
a depth-sensing camera disposed proximate the x-ray source and configured to measure a distance between the x-ray source and the subject accommodated between the x-ray source and x-ray detector; and
one or more processors configured to:
   receive the signal generated by the x-ray detector and generate image data corresponding to a radiographic image of the anatomic feature in the subject based on the received signals; and
   derive one or more dimensions of the anatomic feature based at least in part on the distance between the subject and the radiation source.

Clause 19. The system of Clause 18, wherein the one or more processors are configured generate a map of distances between the x-ray source to different points the subject and co-register at least a portion of the map with the radiographic image of the anatomic feature.

Clause 20. The system of Clause 18 or 19, further comprising an optical camera disposed proximate the radiation source and configured to generate an optical image of at least a portion of the subject, wherein the one or more processors are further configured to generate a composite image of the optical image and the radiographic image.

The examples described herein can be employed using software, hardware, or a combination of software and hardware to implement and perform the systems and methods disclosed herein. Although specific devices have been recited throughout the disclosure as performing specific functions, one of skill in the art will appreciate that these devices are provided for illustrative purposes, and other devices can be employed to perform the functionality disclosed herein without departing from the scope of the disclosure.

This disclosure described some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although specific examples were described herein, the scope of the technology is not limited to those specific examples. One skilled in the art will recognize other examples or improvements that are within the scope of the present technology. Therefore, the specific structure, acts, or media are disclosed only as illustrative examples. Examples according to the invention may also combine elements or components of those that are disclosed in general but not expressly exemplified in combination, unless otherwise stated herein. The scope of the technology is defined by the following claims and any equivalents therein.

1. A system for determining one or more dimensions of an anatomic feature in a subject, the system comprising:
a first imaging system including a radiation source configured to emit radiation toward a subject containing the anatomic feature, a radiation detector configured to receive radiation in response to the radiation emitted from the radiation source toward the subject and to generate signals indicative of an attribute of the received radiation, and one or more processors configured to receive the signals from the radiation detector and generate image data corresponding to an image of the anatomic feature based on the received signals; and
a second imaging system configured to acquire information relating to a relative position between the subject and the radiation source or radiation detector,
the one or more processors being adapted to derive one or more dimensions of the anatomic feature based on the relative position between the subject and the radiation source or radiation detector.

2. The system of claim 1, wherein:
the first imaging system is an x-ray imaging system, the radiation source comprises an x-ray source, and the radiation detector comprises an x-ray detector, the x-ray source and x-ray detector being disposed to accommodate a subject therebetween;
the x-ray source is configured to emit x-ray energy toward the subject;
the x-ray detector is configured to receive x-ray energy emitted from x-ray source and passed through the subject and generate signals indicative of the attenuation of the x-ray energy by different portions of the subject; and the one or processors are programmed to receive the signal from the x-ray detector and generate an image of the subject, including the anatomic feature in the subject, within a field-of-view.

3. The system of claim 2, wherein the second imaging system includes an optical camera disposed in a predetermined position relative to the x-ray source, the optical camera being configured to measure the distance between the subject and the optical camera, thereby determining the relative position between the subject and the x-ray source or x-ray detector of the x-ray imaging system.

4. The system of claim 3, wherein the one or more processors are further configured to derive one or more dimensions of the anatomic feature based on the size of the image of the anatomic feature and the relative position between the subject and the x-ray source or x-ray detector of the x-ray imaging system acquired from the second imaging system.

5. The system of claim 1, wherein the second imaging system includes a second optical camera configured to acquire optical images of the subject.

6. The system of claim 2, further comprising an x-ray collimator for the x-ray source, wherein the optical camera is disposed proximate the x-ray collimator.

7. The system of claim 1, wherein the x-ray imaging system comprises a C-arm x-ray imaging system and further comprises:
a base; and
a C-arm having two extremities and a mid-section between the two extremities, the C-arm being supported by the base at the mid-section,
wherein the x-ray source and x-ray detector are supported at the respective extremities of the C-arm.

8. The system of claim 6, wherein the one or more processors are configured combine the optical images with the images generated by the first imaging system to create composite images.

9. A method for measuring a one or more dimensions of an anatomic feature of a subject, the method including:
acquiring a radiographic image of the anatomic feature using a radiographic imaging system;
determining, substantially simultaneously with the acquisition of the radiographic image, and using an optical imaging system, the position of the subject relative to one or more components of the radiographic imaging system; and
determining the one or more dimensions of the anatomic feature based on the radiographic image and the position of the subject relative to one or more components of the radiographic imaging system.

10. The method of claim 9, wherein the position of the subject relative to one or more components of the radiographic imaging system is measured based on the distance between the subject and the radiation source or detector of the radiographic imaging system.

11. The method of claim 9, wherein the determination of the one or more dimensions of the anatomic feature includes determining the size of the image of the anatomic feature in the radiographic image, and adjusting the size of the image of the anatomic feature by a magnification factor, which is a function of the distance between the subject and one or more components of the radiographic imaging system and of the distance between two or more components of the radiographic imaging system.

12. The system of claim 1, wherein the first imaging system comprises an x-ray imaging apparatus, and the second imaging system comprises a depth-sensing camera disposed and adapted to provide a measure of a source-to-object distance (SOD) between the radiation source and the subject.

13. The system of claim 12, wherein the radiation detector is spaced apart from the radiation source by a source-to-image distance (SID), and the one or more processors are adapted to derive one or more dimensions of the anatomic feature based on SOD, SID, and a size of the image of the anatomic feature.

14. The system of claim 5, wherein the one or more processors are adapted to generate image data corresponding to a composite image of the optical image and the image based on the received signals from the x-ray imaging apparatus.

15. The system of claim 5, further comprising a display adapted to display in real time the optical image acquired by the second optical camera and the image based on the received signals from the x-ray imaging apparatus.

16. The system of claim 11, wherein the depth-sensing camera is adapted to generate a map indicative of distances from the radiation source to respective portions of the subject being imaged.

17. The system of claim 16, wherein the one or more processors are further adapted to co-register the map with the image based on the received signals from the x-ray imaging apparatus.

18. A system for determining a one or more dimensions of an anatomic feature in a subject, the system comprising:
a base;
a C-arm having two extremities and a mid-section between the two extremities, the C-arm being supported by the base at the mid-section,
an x-ray source supported at a first one of the extremities of the C-arm;
an x-ray detector supported at a second one of the extremities of the C-arm,
the x-ray source and x-ray detector being spaced apart to accommodate a subject therebetween the x-ray source being configured to emit x-ray radiation toward the x-ray detector, the x-ray detector being configured to receive the x-ray radiation from the x-ray source and generate signals indicative of an attribute of the received radiation;
a depth-sensing camera disposed proximate the x-ray source and configured to measure a distance between the x-ray source and the subject accommodated between the x-ray source and x-ray detector; and
one or more processors configured to:
receive the signal generated by the x-ray detector and generate image data corresponding to a radiographic image of the anatomic feature in the subject based on the received signals; and
derive one or more dimensions of the anatomic feature based at least in part on the distance between the subject and the radiation source.

19. The system of claim 18, wherein the one or more processors are configured generate a map of distances between the x-ray source to different points the subject and co-register at least a portion of the map with the radiographic image of the anatomic feature.

20. The system of claim 18, further comprising an optical camera disposed proximate the radiation source and configured to generate an optical image of at least a portion of the subject, wherein the one or more processors are further configured to generate a composite image of the optical image and the radiographic image.
